(19) United States
(12) Patent Application Publication
(10) Pub. No.: US 2009/0254072 A1
(43) Pub. Date: Oct. 8, 2009

(54) Laser Catheter with an Adjustable Distal Tip for Increasing the Laser Target Zone

(76) Inventors: Yazan Khatib, Jacksonville, FL (US); Mays Khatib, Jacksonville, FL (US); Ahmed Khatib, Jacksonville, FL (US)

Correspondence Address: AKERMAN SENTERFITT, P.O. BOX 3188, WEST PALM BEACH, FL, 33402-3188, US

(21) Appl. No.: 12/416,718

(22) Filed: Apr. 1, 2009

Related U.S. Application Data
(60) Provisional application No. 61/041,724, filed on Apr. 2, 2008.
(60) Provisional application No. 61/056,650, filed on May 28, 2008.
(60) Provisional application No. 61/056,672, filed on May 28, 2008.

(51) Int. Cl. A61B 18/24

(52) U.S. Cl. 606/7; 606/15

(57) ABSTRACT

A laser catheter having a compliant balloon and a plurality of optical fibers extending from a base to a tip of the catheter for plaque removal is disclosed. The laser catheter may include a distal flush lumen extending to the tip. The compliant balloon may extend along a longitudinal axis of the laser catheter and may be positioned radially outward from an inner lumen. A plurality of optical fibers may be positioned between the inner lumen and an outer compliant material jacket. In another embodiment, the compliant balloon may be positioned eccentrically with respect to the inner lumen. The eccentrically positioned compliant balloon may further facilitate removal of plaque within arteries.

### CROSS-REFERENCE TO RELATED PATENT APPLICATIONS

This patent application claims the benefit of U.S. Provisional Patent Application No. 61/041,724, filed Apr. 2, 2008, U.S. Provisional Patent Application No. 61/056,650, filed May 28, 2008, and U.S. Provisional Patent Application No. 61/056,672, filed May 28, 2008.

### FIELD OF THE INVENTION

This invention is directed generally to catheters, and more particularly to catheters that incorporate lasers for plaque removal.

### BACKGROUND

Some conventional catheters include lasers that are intended to ablate plaque in narrowed vessels in the human body, thus re-establishing normal blood flow. These catheters are typically sized to fit within blood vessels in the human body and remove plaque by striking the plaque with laser beams emitted by the lasers. One such catheter is the Turbo Elite laser catheter by Spectranetics of Colorado Springs, Colo., as illustrated in FIG. 1. Even though the Turbo Elite catheter is approved by the FDA for this function, the device has had limited applicability and utility due to many short comings, not the least of which is the inability to open up large vessels effectively without requiring an excessive amount of procedure time. The function of the catheter is also limited because the catheter can only remove plaque on contact (or in close proximity) to the laser at the tip of the catheter, thus requiring large catheters to be used to effectively clean the blood vessels.

Typically, different catheter diameter sizes are manufactured to accommodate the different size blood vessels found in the human body. For instance, catheters may be manufactured in different vessel sizes ranging between 0.9 mm diameter and 2.5 mm diameter. Catheters on the larger end of this range have been used to clean larger vessels more effectively than smaller catheters. Such is the case because the larger tip on a large catheter has a larger diameter from which laser energy may be emitted to contact plaque on the vessel wall. However, conventional catheters typically have tips that are equivalent in diameter to the catheter shaft. Such a configuration has proven problematic because the entry hole must be as large as the site in the vessel from which plaque is to be removed. This is problematic because the necessity for a larger entry hole creates more potential for vessel trauma and related complications. In addition, in small female patients, a catheter that is large enough to complete the surgery often times will simply not fit through vessel at the entry point (the access site).

An alternative catheter was invented in an attempt to overcome these problems. The alternative catheter, as shown in FIG. 4, includes a laser tip positioned eccentrically within the catheter tip. In such a position, the catheter may be rotated within the vessel to create a larger opening in the vessel than a conventional catheter of the same size and having a concentrically positioned laser. While a first glance this device appears to be an improvement over the catheter first described above, this catheter has proven to be somewhat cumbersome and quite time consuming to use.

### SUMMARY OF THE INVENTION

This invention is directed to a laser catheter with an operationally adjustable laser target zone. The laser catheter may include one or more optical fibers at a tip of the catheter. The laser catheter may be constructed such that the operational laser target zone is variable, thereby enabling the catheter to be inserted into a vessel of a patient where the tip may be enlarged during the process to effectively remove plaque causing arterial blockages by positioning laser emitting optical fibers closer to the walls of the vessel in a patient. The variability of the operational laser target zone enables plaque to be ablated from a vessel more efficiently and in less time than conventional systems.

In another embodiment, the laser catheter may be constructed such that the operational laser target zone is variable and amenable to gradual increments in target ablation. The catheter may also be configured such that directional increments in a target zone can be achieved, thereby enabling the catheter to be inserted into a vessel of a patient such that the tip may be shifted from a central location in a vessel lumen by inflating the eccentrically placed balloon on the side of the tip of the catheter. Such a system enables directional ablation in the areas of eccentric plaque build up. The laser catheter also facilitates more effective removal of plaque causing arterial blockages by positioning laser emitting optical fibers closer to the walls of the vessel in a patient. The variability of the operational laser target zone enables plaque to be ablated from a vessel more efficiently and in less time than conventional systems. The variability of the operational laser target zone also enables the laser energy to be directed where it is most needed in the vessels with eccentric plaques. The eccentrically positioned balloon enables a single catheter to be used to treat multiple sized vessels without the need to use multiple sized catheters.

In one embodiment, the laser catheter may include with an operationally adjustable laser target zone formed from an inner lumen formed by at least one hollow wire and a compliant balloon positioned at least proximate to a tip of the inner lumen such that the compliant balloon is positioned radially outward from the inner lumen. The laser catheter may also include a compliant material jacket positioned radially outward from the compliant balloon that forms an outer housing for the laser catheter at least at the tip and a plurality of optical fibers positioned in the compliant material jacket radially outward from the compliant balloon. The optical fibers may be configured to be placed in communication with at least one laser generator and extend to the tip. The optical fibers terminate at an end of the laser catheter. The laser catheter may also include a distal flush lumen that terminates at a distal end of the laser catheter. The distal flush lumen is eccentrically positioned.

In another embodiment, the laser catheter may include a compliant balloon positioned at least proximate to a tip of the inner lumen such that the compliant balloon is positioned radially outward from the inner lumen and is positioned eccentrically relative to the inner lumen. The eccentric balloon may be attached to an outer surface of the inner lumen and may extend radially outward therefrom. Alternatively, the eccentric balloon may be attached to the inner lumen and extends radially inward therefrom. A distal flush lumen may be included and may terminate at a distal end of the laser catheter, The distal flush lumen may be eccentrically positioned.

An advantage of this invention is that the laser catheter has the ability to change the distal catheter tip diameter after introducing the catheter into the vessel while maintaining a relatively small catheter shaft and thus a small vascular entry point and while maintaining the same centric ablative path. In embodiments in which there is an eccentrically positioned balloon, the orientation of the optical fibers within the tip may be changed. Such orientation will allow an operator physician to define and adjust the desired degree of eccentricity for each particular plaque allowing for example a two millimeter laser catheter to be used to ablate eccentric plaque in vessels as big as 3-8 mm in diameter or larger depending on the inflated diameter used. Such configuration significantly enhances the safety of the device and improves the cost effectiveness by enabling a physician to use one catheter to treat more than one vessel size in one operative session.

Another advantage of this invention is that use of the laser catheter enables one to maintain a relatively small access point sheath size, such as about less than 7 French, whereby each French size is equal to 0.33 mm.

Yet another advantage of this invention is that the laser catheter improves the ease of use of the device.

Another advantage of the laser catheter is that with balloon inflations, the outer surface of the compliant material jacket may touch the vessel wall proximal to the laser ablation site, thereby making the tip more reliable in treating a portion of the vessel at the plaque site such that the site is void of blood and increasing the effectiveness of laser ablation.

Still another advantage of this invention is that the laser catheter may be very useful because of the staggering growth in prevalence of arterial blockages and because of an increasing number of patients with previously implanted stents that have re-occluded due to recurrent plaque.

Another advantage of this invention is that the laser catheter 10 may be very useful because of the staggering growth in prevalence of arterial blockages, and of increasing number of patients with previously implanted stents that have re-occluded due to recurrent plaque.

These and other embodiments are described in more detail below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 is a perspective view of a conventional catheter.

FIG. 2 is a partial perspective view of an inner wire lumen of the catheter of FIG. 1.

FIG. 3 is a perspective view of a tip of the catheter of FIG. 1.

FIG. 4 is a diagram of the path of the laser of a catheter with an eccentrically positioned laser.

FIG. 5 is a cross-sectional view of the catheter of FIG. 1 in a vessel.

FIG. 6A is a cross-sectional side view of a catheter of this invention in a deflated state.

FIG. 6B is a cross-sectional end view of a catheter of this invention in a deflated state.

FIG. 7A is a cross-sectional side view of a catheter of FIG. 6A in an inflated state.

FIG. 7B is a cross-sectional end view of a catheter of this invention in a deflated state.

FIG. 8 is a cross-sectional view of the catheter of FIG. 6A taken orthogonal to a longitudinal axis of the catheter with the balloon inflated along section line 8-8.

FIG. 9 is a cross-sectional view of the catheter of FIG. 7A taken orthogonal to a longitudinal axis of the catheter with the balloon deflated along section line 9-9.

FIG. 10A is a cross-sectional side view of an alternative catheter of this invention in a deflated state including an eccentric wire lumen and centrally positioned distal flush lumen.

FIG. 11 is a cross-sectional view of the catheter of FIG. 10A taken orthogonal to a longitudinal axis of the catheter with the balloon deflated along section line 11-11.

FIG. 12 is a cross-sectional side view of a catheter of this invention in a deflated state.

FIG. 13 is a cross-sectional side view of a catheter of FIG. 12 in an inflated state.

FIG. 14 is a cross-sectional view of the catheter of FIG. 12 taken orthogonal to a longitudinal axis of the catheter with the balloon deflated along section line 14-14.

FIG. 15 is a cross-sectional view of the catheter of FIG. 13 taken orthogonal to a longitudinal axis of the catheter with the balloon inflated along section line 15-15.

FIG. 16 is a cross-sectional view of an alternative catheter of this invention in a inflated state including an eccentric wire lumen and centrally positioned distal flush lumen.

FIG. 17 is a cross-sectional view of the catheter of FIG. 16 taken orthogonal to a longitudinal axis of the catheter with the balloon deflated.

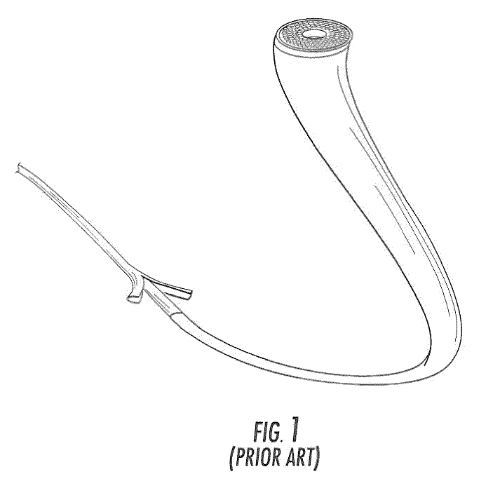

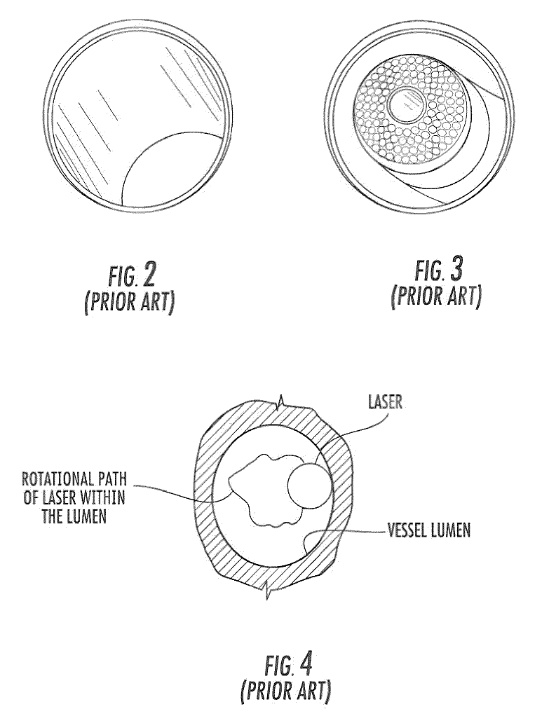

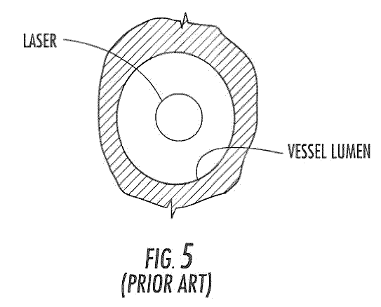

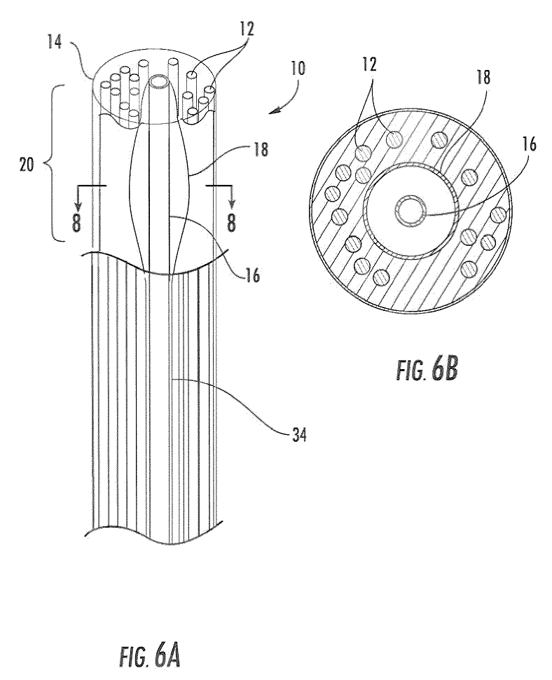

### DETAILED DESCRIPTION OF THE INVENTION

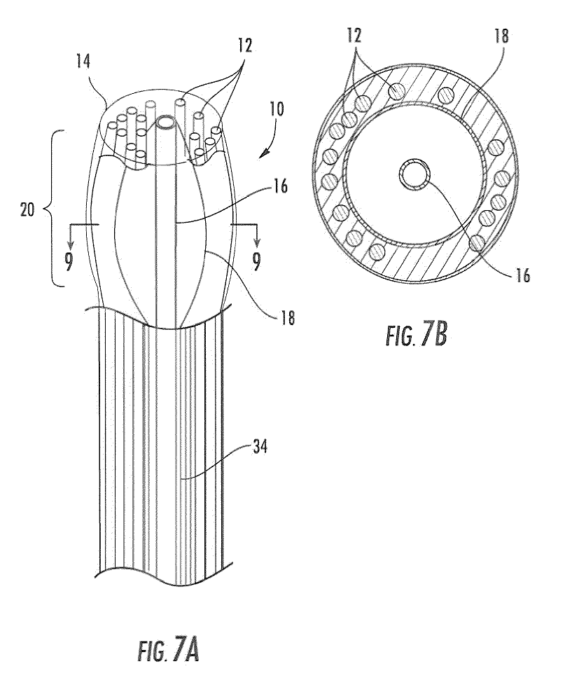

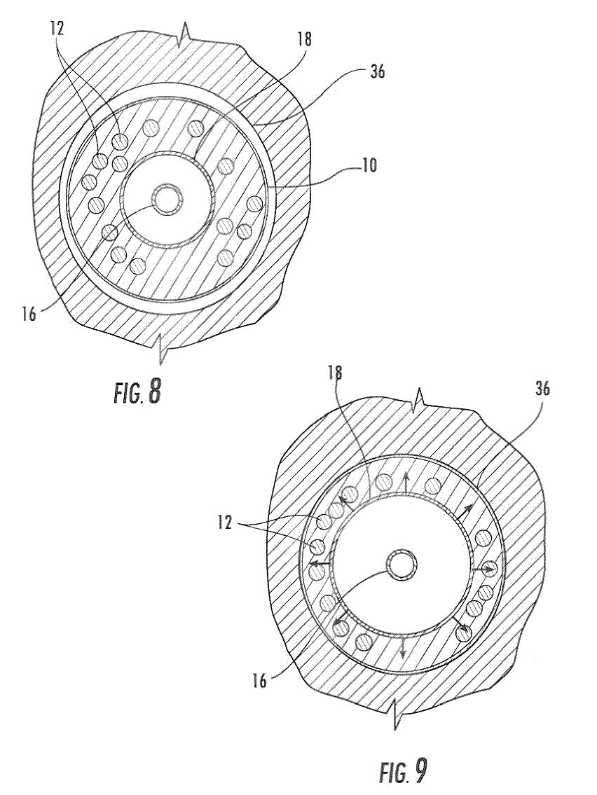

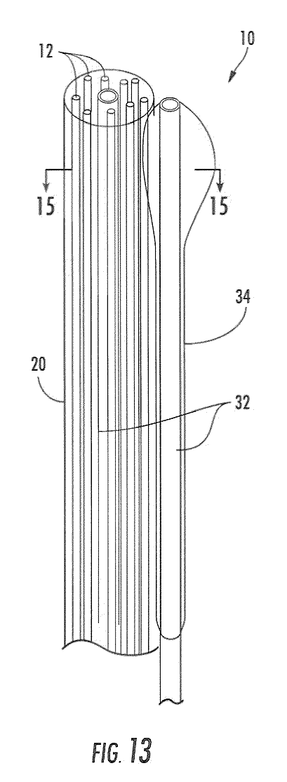

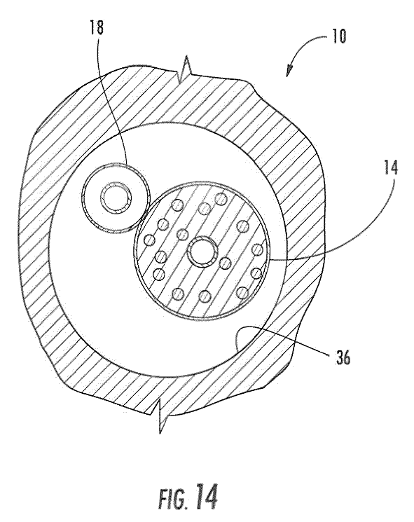

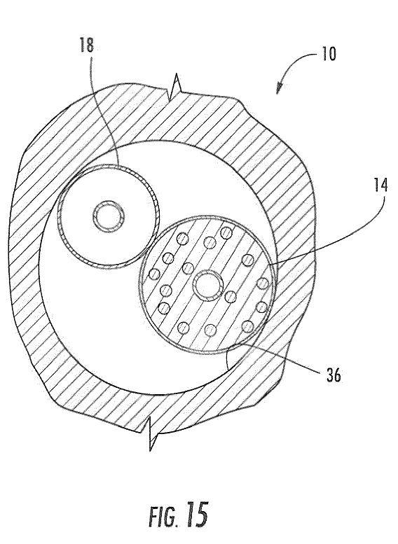

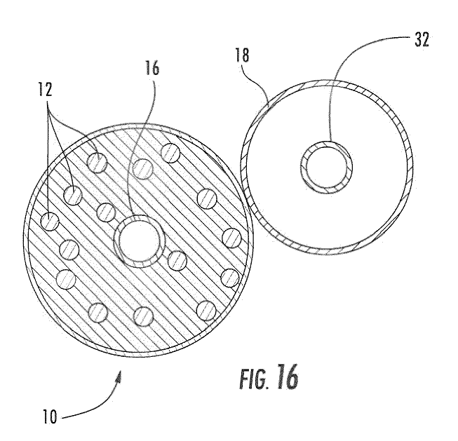

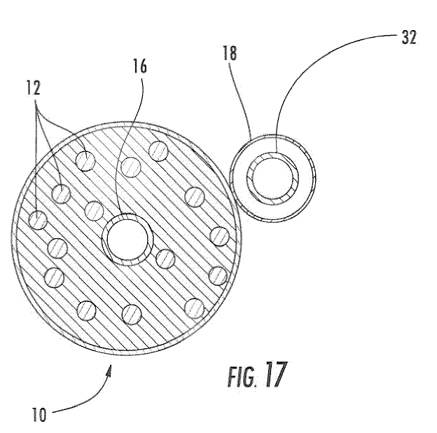

As shown in FIGS. 6-17, this invention is directed to a laser catheter 10 with an operationally adjustable laser target zone. The laser catheter 10 may include one or more optical fibers 12 at a distal tip 14 of the catheter 10. The laser catheter 10 may be constructed such that the operational laser target zone is variable, thereby enabling the catheter 10 to be inserted into a vessel of a patient and then enlarged during the process to effectively remove plaque causing arterial blockages. The variability of the operational laser target zone enables plaque to be ablated from a vessel more efficiently, more safely and in less time than conventional systems.

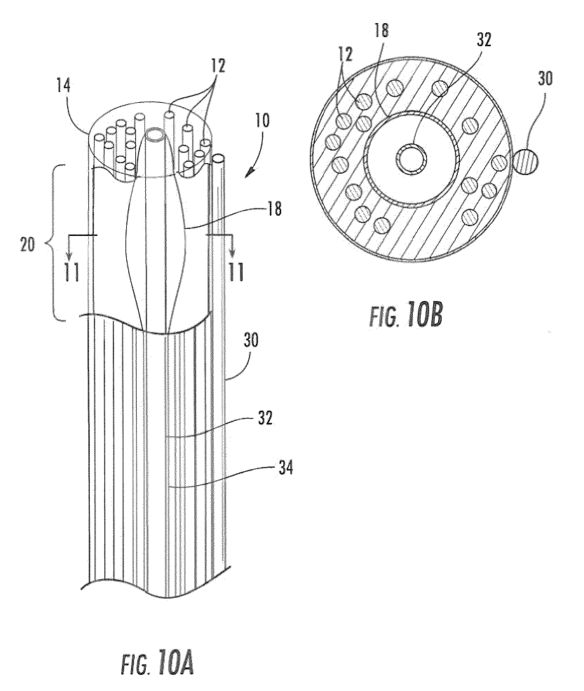

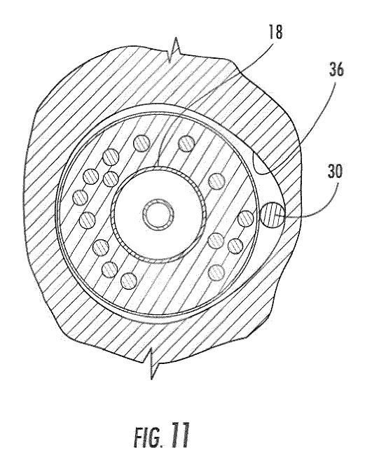

The laser catheter 10 may be formed from a flexible, hollow tube 16, which may be referred to as an inner lumen, as shown in FIGS. 6A-7B. The hollow tube 16 may be formed from any appropriate material and in any appropriate configuration to provide the necessary support together with the necessary flexibility to be inserted into and manipulated within a vessel of a patient. An example of an appropriate hollow tube 16 is included within the laser catheters sold by The Spectranetics Corporation of Colorado Springs, Colo. The hollow tube 16 may also function as a distal flush lumen 32, as shown in FIG. 10, by allowing fluids to be transported in the voids between a wire and the lumen. A compliant balloon 18, such as, but not limited to an over the wire or a rapid exchange compliant balloon, may be positioned proximate to a distal tip 14 of the inner lumen 16 such that the compliant balloon 18 is positioned radially outward from the inner lumen 16. In one embodiment, the compliant balloon 18 may be positioned at or immediately proximate to a distal tip 14 of the inner lumen 16 such that the compliant balloon 18 is positioned radially outward from the inner lumen 16. The balloon 18 may be any appropriate sized balloon formed from any appropriate material. The balloon distal tip may be a long tapered shoulder or may be a no shoulder design. The balloon 18 may be inflated and deflated with a balloon supply lumen 34.

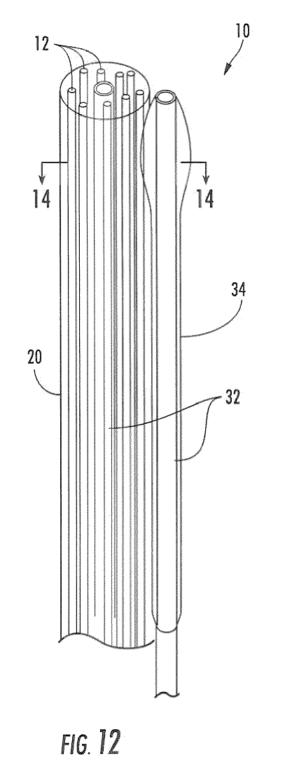

In another embodiment, the compliant balloon 18 may be positioned eccentrically, as shown in FIGS. 12-17. In particular, the compliant balloon 18 may be positioned such that the compliant balloon 18 is positioned eccentrically at inner or outer surfaces of a compliant material jacket 20. The compliant balloon 18 may be of any appropriate shape, including, but not limited to, a crescent shape and other shapes that would facilitate advancement of the compliant balloon into a patient.

In yet another embodiment, the laser catheter 10 may include an eccentric wire lumen 30, as shown in FIGS. 10 and 11, configured to receive a catheter wire. The eccentric wire lumen 30 may be positioned at an outer surface of the laser catheter 10. The eccentric wire lumen 30 may have any appropriate size. The eccentric wire lumen may include a distal flush as well.

The laser catheter 10 may also include a compliant material jacket 20 positioned radially outward from the compliant balloon 18 as shown in FIGS. 6A, 7A, 10A, 12 and 13. The compliant material jacket 20 may form an elongated outer housing for the laser catheter 10. In one embodiment, the inner lumen 16 may be positioned concentrically within the compliant balloon 18, and the compliant balloon 18 may be positioned concentrically within the compliant material jacket 20. The compliant material jacket 20 contains the compliant balloon 18 within the laser catheter 10 yet allows the compliant balloon 18 to inflate within a vessel. During use, in one embodiment, the tip 14 may be about two millimeters in diameter in a deflated state and may be inflated such that an outer diameter of the tip 14 is about 4.5 mm or larger when the balloon is maximally inflated. This size range is exemplary only and is not provided as a limitation of the invention. In other embodiments, the size of the tip 14 in the deflated and inflated states may be greater than or less than the size range provided. In the embodiment in which the compliant balloon 18 is positioned eccentrically, as the compliant balloon 18 is inflated, the optical fibers 12 move into an increasingly greater eccentric position, thereby putting the optical fibers 12 in contact with eccentric plaques in larger vessels.

The laser catheter 10 may include one or more optical fibers 12 positioned in the compliant material jacket 20 that is radially outward from the compliant balloon 18. The optical fibers 12 may be in communication with at least one laser generator (not shown). In at least one embodiment, the laser catheter 10 may include a plurality of optical fibers 12 positioned within the compliant material jacket 20. The optical fibers 12 may extend generally parallel to the inner lumen 16 and may be positioned radially outward from the inner lumen 16. The optical fibers 12 may be positioned circumferentially around the inner lumen 16. The balloon 18 may be positioned centrally within the circular configuration of the inner lumen 16 or eccentrically within the laser catheter 10 such as eccentrically within or immediately radially outside of a catheter sheath. The optical fibers 12 may be spaced equidistant from each other, spaced random distances from each other, positioned in patterns, or positioned otherwise. The optical fibers 12 may terminate at the tip 14 such that laser beams may be emitted from the optical fibers 12 and strike plaque within vessels in a patient. In another embodiment, the optical fibers 12 may be placed around the wire lumen 16 with the distal flush lumen 32 at the tip 14 of the catheter or at a distance from the tip 14.

During use, the catheter 10 of FIGS. 6A-11 may be inserted into the vessel 36 of a patient. Preferably, the outer diameter of the tip 14 is as small as possible to limit the size of the site at which the catheter 10 is inserted. The catheter may be inserted 10 a sufficient distance to place the tip 14 in very close proximity to plaque within the vessel. The laser may be actuated to emit a laser beam from the optical fibers 12 to ablate the plaque buildup in the vessel. After the initial pass has been completed establishing a lumen, the balloon 18 may be inflated such that the outer surface of the compliant material jacket 20 at least nearly contacts the vessel wall other amount depending on the vessel size and the patient needs. The laser may be actuated further to ablate the plaque buildup in the vessel. This process can be repeated as needed with further balloon inflation and catheter rotational manipulation as deemed necessary for each particular point until all desired plaque removal is achieved. A very good benefit of the laser catheter 10 is that with balloon inflations, the outer surface of the compliant material jacket 20 and therefore some of the optical fibers 12 touch or nearly touch the vessel wall proximal to the laser ablation site, thereby positioning the tip 14 in a more central position within the vessel. Such positioning further enhances plaque ablation by making the vessel at the plaque site more void of blood and increases the effectiveness of the laser ablation.

In the embodiment in which the compliant balloon 18 is positioned eccentrically, as shown in FIGS. 12-17, the catheter may be inserted 10 a sufficient distance to place the tip 14 in very close proximity to plaque within the vessel. The laser may be actuated to emit a laser beam from the optical fibers 12 to ablate the plaque buildup in the vessel. After the initial pass has been completed establishing a lumen, the compliant balloon 18 may be inflated such that the laser tip is pushed away from the center of the lumen 16 and positioned eccentrically within the lumen 16, thereby positioning the optical fibers 12 in close proximity to eccentrically positioned plaque.

The foregoing is provided for purposes of illustrating, explaining, and describing embodiments of this invention. Modifications and adaptations to these embodiments will be apparent to those skilled in the art and may be made without departing from the scope or spirit of this invention.

I claim:

1. A laser catheter with an operationally adjustable laser target zone, comprising:
an inner lumen formed by at least one hollow wire;
a compliant balloon positioned at least proximate to a tip of the inner lumen such that the compliant balloon is positioned radially outward from the inner lumen;
a compliant material jacket positioned radially outward from the compliant balloon that forms an elongated outer housing for the laser catheter at least at the tip; and
a plurality of optical fibers positioned in the compliant material jacket radially outward from the compliant balloon, wherein the optical fibers are configured to be placed in communication with at least one laser generator and extend to the tip.

2. The laser catheter of claim 1, wherein the optical fibers terminate at an end of the laser catheter.

3. The laser catheter of claim 1, further comprising a distal flush lumen that terminates at a distal end of the laser catheter.

4. The laser catheter of claim 4, wherein the distal flush lumen is eccentrically positioned.

5. The laser catheter of claim 1, wherein the inner lumen is positioned eccentrically.

6. A laser catheter with an operationally adjustable laser target zone, comprising:
an inner lumen formed by at least one hollow wire;
a compliant balloon positioned at least proximate to a tip of the inner lumen such that the compliant balloon is positioned radially outward from the inner lumen and is positioned eccentrically relative to the inner lumen;
a compliant material jacket positioned radially outward from the compliant balloon that forms an outer housing for the laser catheter at least at the tip; and
a plurality of optical fibers positioned in the compliant material jacket, wherein the optical fibers are configured to be placed in communication with at least one laser generator and extend to the tip.

7. The laser catheter of claim 6, wherein the eccentric balloon is attached to an outer surface of the inner lumen and extends radially outward therefrom.

8. The laser catheter of claim 6, wherein the eccentric balloon is attached to the inner lumen and extends radially inward therefrom.

9. The laser catheter of claim 6, further comprising a distal flush lumen that terminates at a distal end of the laser catheter.

10. The laser catheter of claim 9, wherein the distal flush lumen is eccentrically positioned.

11. The laser catheter of claim 6, wherein the inner lumen is positioned eccentrically.
